(19) United States
(12) Patent Application Publication
(10) Pub. No.: US 2014/0148477 A1
(43) Pub. Date: May 29, 2014

(54) ANIMAL ECTOPARASITE-CONTROLLING AGENT AND METHOD FOR PREVENTING OR TREATING INFECTION IN ANIMALS CAUSED BY PARASITES BY USING THE CONTROLLING AGENT

(71) Applicants: Yasuhiro Endo, Naruto-shi (JP); Yuichi Shirai, Naruto-shi (JP)

(72) Inventors: Yasuhiro Endo, Naruto-shi (JP); Yuichi Shirai, Naruto-shi (JP)

(73) Assignee: OTSUKA AGRITECHNO CO., LTD., Tokyo (JP)

(21) Appl. No.: 14/128,807

(22) PCT Filed: Jul. 19, 2012
(86) PCT No.: PCT/JP2012/069263

§ 371 Date: Dec. 23, 2013

(30) Foreign Application Priority Data
Jul. 26, 2011 (JP) 2011-163304

(51) Int. Cl. A61K 31/4545; A61K 31/439

(52) U.S. Cl. CPC A61K 31/4545; A61K 31/439
USPC 514/299; 546/193; 546/194; 546/183; 514/318

(57) ABSTRACT

The present invention provides an animal ectoparasite-controlling agent and a method for preventing or treating infection in animals caused by parasites by using the controlling agent. An animal ectoparasite-controlling agent exhibiting excellent insecticidal activity, and a method for preventing or treating infection in animals caused by parasites by using the controlling agent are obtained by using a compound having a pyrazole ring at the 4-position of the piperidine ring as an active ingredient.

### TECHNICAL FIELD

The present invention relates to an animal ectoparasite-controlling agent comprising an N-pyridylpiperidine compound as an active ingredient, and to a method for preventing or treating infection in animals caused by parasites by using the controlling agent.

### BACKGROUND ART

Various controlling agents and repellents for ectoparasites of animals have been developed so far; however, there is always the possibility that a species having resistance to the active ingredients of such agents may appear. For this reason, there is a constant demand for research and development of novel active ingredients.

Meanwhile, an N-pyridylpiperidine compound, which is an active ingredient of the present invention, has already been reported (see PTL 1). This document discloses that the N-pyridylpiperidine compound exhibits miticidal activity against plant-parasitic mites. However, the insecticidal effect of the N-pyridylpiperidine compound on animal ectoparasites was not known at all.

### CITATION LIST

### Patent Literature

PTL 1: WO 2008/026658

### SUMMARY OF INVENTION

### Technical Problem

The present invention has been made in view of the above circumstances, and an object of the present invention is to provide an animal ectoparasite-controlling agent, and a method for preventing or treating infection in animals caused by parasites by using the controlling agent.

### Solution to Problem

The present inventors conducted extensive research to achieve the above object and found that the compound disclosed in PTL 1 having a pyrazole ring at the 4-position of the piperidine ring also exhibited excellent insecticidal activity against animal ectoparasites. The present invention has been accomplished based on this finding.

More specifically, the present invention provides an animal ectoparasite-controlling agent, and a method for preventing or treating infection in animals caused by parasites by using the controlling agent, as summarized below. Item 1. An animal ectoparasite-controlling agent comprising an N-pyridylpiperidine compound, an N-oxide thereof, or salts of these compounds, the N-pyridylpiperidine compound being represented by Formula (1):

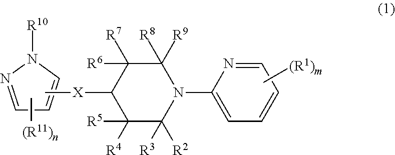

wherein R¹ is a halogen atom, a C₁₋₄ haloalkyl group, a cyano group, a nitro group, or a C₁₋₄ alkoxycarbonyl group;

R², R³, R⁴, R⁵, R⁶, R⁷, R⁸, and R⁹ are each a hydrogen atom or a C₁₋₄ alkyl group;

each pair of R² and R⁸, and R⁴ and R⁶, may join to form a C₁₋₄ alkylene group;

R¹⁰ is a hydrogen atom; a C₁₋₂₀ alkyl group; a C₃₋₈ cycloalkyl group; a C₂₋₆ alkenyl group; a C₂₋₆ alkynyl group; a C₁₋₆ haloalkyl group; a C₂₋₆ haloalkenyl group; a C₁₋₆ alkylcarbonyl group; a C₁₋₆ alkoxycarbonyl group; a benzoyl group optionally substituted on the phenyl ring with one to five halogen atoms; a phenyl group optionally substituted on the phenyl ring with one or more substituents each independently selected from the group consisting of halogen, C₁₋₄ alkyl, and C₁₋₄ haloalkyl; a heterocyclic group optionally substituted on the heterocyclic ring with one or more substituents each independently selected from the group consisting of halogen, C₁₋₄ alkyl, C₁₋₄ haloalkyl, and optionally substituted heterocyclic groups; or a C₁₋₄ alkyl group optionally substituted with one or more substituents each independently selected from the group consisting of optionally halogen-substituted C₃₋₈ cycloalkyl, cyano, nitro, formyl, C₁₋₆ alkoxy, C₁₋₄ haloalkoxy, benzyloxy, phenoxy, —CON(R¹²)(R¹³), phenyl optionally substituted on the phenyl ring with one or more halogen atoms, and heterocyclic groups optionally substituted on the heterocyclic ring with one or more C₁₋₄ alkyl groups; wherein R¹² and R¹³ are each a C₁₋₄ alkyl group, or R¹² and R¹³ may join to form a C₂₋₇ alkylene group;

R¹¹ is a halogen atom; a C₁₋₆ alkyl group; a C₁₋₄ haloalkyl group; a C₁₋₄ hydroxyalkyl group; a C₁₋₄ alkoxycarbonyl group; a C₁₋₄ alkylcarbonyl group; a mono or di(C₁₋₄ alkyl)aminocarbonyl group; a nitro group; a cyano group; a formyl group; —C(R¹⁴)═NO(R¹⁵); a phenyl group optionally substituted on the phenyl ring with one or more substituents each independently selected from the group consisting of halogen, C₁₋₆ alkyl, C₁₋₄ haloalkyl, C₁₋₆ alkoxy, C₁₋₄ haloalkoxy, C₁₋₄ alkylthio, cyano, and nitro; or a heterocyclic group optionally substituted on the heterocyclic ring with one or more substituents each independently selected from the group consisting of halogen, C₁₋₄ alkyl, and C₁₋₄ haloalkyl; wherein R¹⁴ is a hydrogen atom or a C₁₋₄ alkyl group, and R¹⁵ is a hydrogen atom, a C₁₋₄ alkyl group, or a benzyl group;

X is an oxygen atom, a sulfur atom, or —SO₂—;

m is an integer of 1 to 4, and when m is an integer of 2 or more, the R¹'s, the number of which is represented by m, may be the same or different; and

n is an integer of 1 or 2, and when n is 2, the two R¹¹'s may be the same or different.
Item 2. The animal ectoparasite-controlling agent according to Item 1, wherein the N-pyridylpiperidine compound is represented by Formula (1) in which R¹ is a halogen atom, a C₁₋₄ haloalkyl group, a cyano group, or a nitro group.
Item 3. The animal ectoparasite-controlling agent according to Item 1, wherein the N-pyridylpiperidine compound is represented by Formula (1) in which R¹⁰ is a hydrogen atom; a C₁₋₂₀ alkyl group; a C₂₋₆ alkenyl group; a C₁₋₆ haloalkyl group; a C₁₋₆ alkylcarbonyl group; a C₁₋₆ alkoxycarbonyl group; a benzoyl group optionally substituted on the phenyl ring with one to five halogen atoms; a phenyl group optionally substituted on the phenyl ring with one or more substituents each independently selected from the group consisting of halogen, C₁₋₄ alkyl, and C₁₋₄ haloalkyl; a heterocyclic group optionally substituted on the heterocyclic ring with one or more substituents each independently selected from the group consisting of halogen, C₁₋₄ alkyl, C₁₋₄ haloalkyl, and optionally substituted heterocyclic groups; or a C₁₋₄ alkyl group substituted with one or more substituents each independently selected from the group consisting of formyl, C₁₋₆ alkoxy, phenyl optionally substituted on the phenyl ring with one or more halogen atoms, and heterocyclic groups optionally substituted on the heterocyclic ring with one or more C₁₋₄ alkyl groups.
Item 4. The animal ectoparasite-controlling agent according to Item 1, wherein the N-pyridylpiperidine compound is represented by Formula (1) in which R¹¹ is a halogen atom; a C₁₋₆ alkyl group; a C₁₋₄ haloalkyl group; a C₁₋₄ hydroxyalkyl group; a C₁₋₄ alkoxycarbonyl group; a formyl group; —C(R¹⁴)═NO(R¹⁵) wherein R¹⁴ is a hydrogen atom, and R¹⁵ is a hydrogen atom or a C₁₋₄ alkyl group; a phenyl group optionally substituted on the phenyl ring with one or more substituents each independently selected from the group consisting of halogen, C₁₋₆ alkyl, C₁₋₄ haloalkyl, C₁₋₆ alkoxy, C₁₋₄ haloalkoxy, C₁₋₄ alkylthio, cyano, and nitro; or a heterocyclic group optionally substituted on the heterocyclic ring with one or more halogen atoms.
Item 5. The animal ectoparasite-controlling agent according to Item 1, wherein the N-pyridylpiperidine compound is represented by Formula (1) in which X is an oxygen atom.
Item 6. A method for preventing or treating infection in an animal caused by parasites, the method comprising administrating the animal ectoparasite-controlling agent according to any one of Items 1 to 5 to the animal.

### Advantageous Effect of Invention

The present invention can provide an animal ectoparasite-controlling agent having an excellent control effect on animal ectoparasites, such as mites.

### DESCRIPTION OF EMBODIMENTS

The present invention is described in detail below.

### N-pyridylpiperidine Compound

The controlling agent of the present invention comprises, as an active ingredient, a compound represented by the following Formula (1) and having pyrazole bonded to the 4-position of the piperidine ring via an oxygen or sulfur atom.

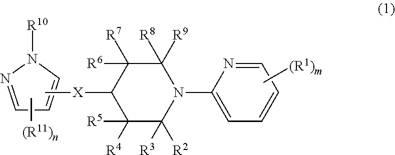

wherein R¹ is a halogen atom, a C₁₋₄ haloalkyl group, a cyano group, a nitro group, or a C₁₋₄ alkoxycarbonyl group;

R², R³, R⁴, R⁵, R⁶, R⁷, R⁸, and R⁹ are each a hydrogen atom or a C₁₋₄ alkyl group;

each pair of R² and R⁸, and R⁴ and R⁶, may join to form a C₁₋₄ alkylene group;

R¹⁰ is a hydrogen atom; a C₁₋₂₀ alkyl group; a C₃₋₈ cycloalkyl group; a C₂₋₆ alkenyl group; a C₂₋₆ alkynyl group; a C₁₋₆ haloalkyl group; a C₂₋₆ haloalkenyl group; a C₁₋₆ alkylcarbonyl group; a C₁₋₆ alkoxycarbonyl group; a benzoyl group optionally substituted on the phenyl ring with 1 to 5 halogen atoms; a phenyl group optionally substituted on the phenyl ring with one or more substituents each independently selected from the group consisting of halogen, C₁₋₄ alkyl, and C₁₋₄ haloalkyl; a heterocyclic group optionally substituted on the heterocyclic ring with one or more substituents each independently selected from the group consisting of halogen, C₁₋₄ alkyl, C₁₋₄ haloalkyl, and optionally substituted heterocyclic groups; or a C₁₋₄ alkyl group optionally substituted with one or more substituents each independently selected from the group consisting of optionally halogen-substituted C₃₋₈ cycloalkyl, cyano, nitro, formyl, C₁₋₆ alkoxy, C₁₋₄ haloalkoxy, benzyloxy, phenoxy, —CON(R¹²)(R¹³), phenyl optionally substituted on the phenyl ring with one or more halogen atoms, and heterocyclic groups optionally substituted on the heterocyclic ring with one or more C₁₋₄ alkyl groups; wherein R¹² and R¹³ are each a C₁₋₄ alkyl group, or R¹² and R¹³ may join to form a C₂₋₇ alkylene group;

R¹¹ is a halogen atom; a C₁₋₆ alkyl group; a C₁₋₄ haloalkyl group; a C₁₋₄ hydroxyalkyl group; a C₁₋₄ alkoxycarbonyl group; a C₁₋₄ alkylcarbonyl group; a mono or di(C₁₋₄ alkyl)aminocarbonyl group; a nitro group; a cyano group; a formyl group; —C(R¹⁴)═NO(R¹⁵); a phenyl group optionally substituted on the phenyl ring with one or more substituents each independently selected from the group consisting of halogen, C₁₋₆ alkyl, C₁₋₄ haloalkyl, C₁₋₆ alkoxy, C₁₋₄ haloalkoxy, C₁₋₄ alkylthio, cyano, and nitro; or a heterocyclic group optionally substituted on the heterocyclic ring with one or more substituents each independently selected from the group consisting of halogen, C₁₋₄ alkyl, and C₁₋₄ haloalkyl; wherein R¹⁴ is a hydrogen atom or a C₁₋₄ alkyl group, and R¹⁵ is a hydrogen atom, a C₁₋₄ alkyl group, or a benzyl group;

X is an oxygen atom, a sulfur atom, or —SO₂—;

m is an integer of 1 to 4, and when m is an integer of 2 or more, the R¹'s, the number of which is represented by m, may be the same or different; and

n is an integer of 1 or 2, and when n is 2, the two R¹¹'s may be the same or different.

Examples of the halogen atom include fluorine, chlorine, bromine, and iodine atoms.

Examples of the C₁₋₄ haloalkyl group include linear or branched alkyl groups having 1 to 4 carbon atoms and substituted with 1 to 9, preferably 1 to 5, halogen atoms. Specific examples thereof include fluoromethyl, chloromethyl, bromomethyl, iodomethyl, difluoromethyl, trifluoromethyl, chlorodifluoromethyl, bromodifluoromethyl, dichlorofluoromethyl, 1-fluoroethyl, 2-fluoroethyl, 2-chloroethyl, 2-bromoethyl, 2-iodoethyl, 2,2,2-trifluoroethyl, 2,2,2-trichloroethyl, pentafluoroethyl, 1-fluoroisopropyl, 3-fluoropropyl, 3-chloropropyl, 3-bromopropyl, 4-fluorobutyl, 4-chlorobutyl, 4,4,4-trifluorobutyl, and like groups.

Examples of the C₁₋₄ alkoxycarbonyl group include groups formed by the bonding of a linear or branched alkoxy group having 1 to 4 carbon atoms to a carbonyl group. Specific examples thereof include methoxycarbonyl, ethoxycarbonyl, n-propoxycarbonyl, isopropoxycarbonyl, n-butoxycarbonyl, sec-butoxycarbonyl, tert-butoxycarbonyl, and like groups.

Examples of the C₁₋₄ alkyl group include linear or branched alkyl groups having 1 to 4 carbon atoms, such as methyl, ethyl, n-propyl, isopropyl, n-butyl, isobutyl, sec-butyl, and tert-butyl.

Examples of the C₁₋₄ alkylene group include linear or branched alkylene groups having 1 to 4 carbon atoms, such as methylene, ethylene, trimethylene, tetramethylene, propylene, and ethylethylene.

Examples of the C₁₋₆ alkyl group include linear or branched alkyl groups having 1 to 6 carbon atoms, such as n-pentyl, isopentyl, neopentyl, tert-pentyl, n-hexyl, isohexyl, and 2-ethyl-n-butyl, in addition to those mentioned as examples of the C₁₋₄ alkyl group.

Examples of the C₁₋₂₀ alkyl group include linear or branched alkyl groups having 1 to 20 carbon atoms, such as n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl, in addition to those mentioned as examples of the C₁₋₄ alkyl group and C₁₋₆ alkyl group.

Examples of the C₃₋₈ cycloalkyl group include cyclic alkyl groups having 4 to 8 carbon atoms, such as cyclopropyl, cyclobutyl, cyclopentyl, cyclohexyl, cycloheptyl, and cyclooctyl.

Examples of the C₂₋₆ alkenyl group include linear or branched alkenyl groups containing 2 to 6 carbon atoms and having at least one double bond at any position. Specific examples thereof include vinyl, 1-propenyl, allyl, isopropenyl, 2-butenyl, 3-butenyl, 1-methyl-2-propenyl, 1,3-butadienyl, 1-pentenyl, 2-pentenyl, 3-pentenyl, 4-pentenyl, 1,1-dimethyl-2-propenyl, 1-ethyl-2-propenyl, 1-methyl-2-butenyl, 1-methyl-3-butenyl, 1-hexenyl, 2-hexenyl, 3-hexenyl, 4-hexenyl, 5-hexenyl, 1,1-dimethyl-2-butenyl, 1,1-dimethyl-3-butenyl, and like groups.

Examples of the C₂₋₆ alkynyl group include linear or branched alkynyl groups containing 2 to 6 carbon atoms and having at least one triple bond at any position. Specific examples thereof include ethynyl, 2-propynyl, 1-methyl-2-propynyl, 1,1-dimethyl-2-propynyl, 1-butynyl, 2-butynyl, 3-butynyl, 1-pentynyl, 2-pentynyl, 3-pentynyl, 4-pentynyl, 1-methyl-2-butynyl, 1-methyl-3-butynyl, 1,1-dimethyl-2-butynyl, 1,1-dimethyl-3-butynyl, 1-methyl-3-pentynyl, 1-methyl-4-pentynyl, and like groups.

Examples of the C₁₋₆ haloalkyl group include linear or branched alkyl groups having 1 to 6 carbon atoms and substituted with 1 to 13, preferably 1 to 7, halogen atoms. Specific examples thereof include 5-chloropentyl, 5-fluoropentyl, 6-chlorohexyl, and 6-fluorohexyl, in addition to those mentioned as examples of the C₁₋₄ haloalkyl group.

Examples of the C₂₋₆ haloalkenyl group include C₂₋₆ linear or branched alkenyl groups having at least one double bond at any position and substituted with 1 to 13, preferably 1 to 7, halogen atoms. Specific examples thereof include 2,2-dichlorovinyl, 2,2-dibromovinyl, 3-chloro-2-propenyl, 3,3-difluoro-2-allyl, 3,3-dichloro-2-allyl, 4-chloro-2-butenyl, 4,4,4-trifluoro-2-butenyl, 4,4,4-trichloro-3-butenyl, 5-chloro-3-pentenyl, 6-fluoro-2-hexenyl, and like groups.

Examples of the heterocyclic group include thienyl, furyl, tetrahydrofuryl, dioxolanyl, dioxanyl, pyrrolyl, pyrrolinyl, pyrrolidinyl, oxazolyl, isoxazolyl, oxazolinyl, oxazolidinyl, isoxazolinyl, triazolyl, isothiazolyl, thiazolinyl, thiazolidinyl, isothiazolinyl, pyrazolyl, pyrazolidinyl, imidazolyl, imidazolinyl, imidazolidinyl, oxadiazolyl, oxadiazolinyl, thiadiazolinyl, triazolyl, triazolinyl, triazolidinyl, tetrazolyl, tetrazolinyl, pyridyl, dihydropyridyl, tetrahydropyridyl, piperidyl, oxazinyl, dihydroxazinyl, morpholino, thiazinyl, dihydrothiazinyl, thiamorpholino, pyridazinyl, dihydropyridazinyl, tetrahydropyridazinyl, hexahydropyridazinyl, oxadiazinyl, dihydrooxadiazinyl, tetrahydrooxadiazinyl, thiadiazolyl, thiadiazinyl, dihydrothiadiazinyl, tetrahydrothiadiazinyl, pyrimidinyl, dihydropyrimidinyl, tetrahydropyrimidinyl, hexahydropyrimidinyl, pyrazinyl, dihydropyrazinyl, tetrahydropyrazinyl, piperazinyl, triazinyl, dihydrotriazinyl, tetrahydrotriazinyl, hexahydrotriazinyl, tetrazinyl, dihydrotetrazinyl, indolyl, indolinyl, isoindolyl, indazolyl, quinazolinyl, dihydroquinazolyl, tetrahydroquinazolyl, carbazolyl, benzoxazolyl, benzoxazolinyl, benzisoxazolyl, benzisoxazolinyl, benzothiazolyl, benzisothiazolyl, benzisothiazolinyl, benzimidazolyl, indazolinyl, quinolinyl, dihydroquinolinyl, tetrahydroquinolinyl, isoquinolinyl, dihydroisoquinolinyl, tetrahydroisoquinolinyl, pyridoindolyl, dihydrobenzoxazinyl, cinnolinyl, dihydrocinnolinyl, tetrahydrocinnolinyl, phthalazinyl, dihydrophthalazinyl, tetrahydrophthalazinyl, quinoxalinyl, dihydroquinoxalinyl, tetrahydroquinoxalinyl, purinyl, dihydrobenzotriazinyl, dihydrobenzotetrazinyl, phenothiazinyl, furanyl, benzofuranyl, benzothienyl, and like groups. These heterocyclic groups include those substituted at any substitutable position with an oxo or thioketone group. These heterocyclic groups further include those optionally substituted at any substitutable position with 1 to 5 (preferably 1 to 3) substituents, such as halogen atoms, C₁₋₄ alkyl groups, C₁₋₄ haloalkyl groups, or substituted heterocyclic groups (e.g., 3-chloropyridin-2-yl, 4-trifluoromethyl-1,3-thiazol-2-yl, and 5-trifluoromethylpyridin-2-yl).

Among these heterocyclic rings, thienyl, furyl, tetrahydrofuryl, dioxolanyl, dioxanyl, oxazolyl, isoxazolyl, thiazolyl, pyrazolyl, pyridyl, and piperidyl are preferable. Thienyl, tetrahydrofuryl, dioxolanyl, dioxanyl, thiazolyl, and pyridyl are particularly preferable.

Examples of the optionally halogen-substituted C₃₋₈ cycloalkyl group include cyclic alkyl groups having 3 to 8 carbon atoms, such as the above-mentioned C₃₋₈ cycloalkyl groups that are optionally substituted at any position with one to the maximum substitutable number of (preferably 1 to 5, and more preferably 1 to 3) halogen atoms.

Examples of the C₁₋₆ alkoxy group include linear or branched alkoxy groups having 1 to 6 carbon atoms, such as methoxy, ethoxy, n-propoxy, isopropoxy, cyclopropyloxy, n-butoxy, sec-butoxy, tert-butoxy, n-pentyloxy, isopentyloxy, neopentyloxy, tert-pentyloxy, n-hexyloxy, and isohexyloxy.

Examples of the C₁₋₄ haloalkoxy group include linear or branched alkoxy groups having 1 to 4 carbon atoms and substituted with 1 to 9, preferably 1 to 5, halogen atoms. Specific examples thereof include fluoromethoxy, chloromethoxy, bromomethoxy, iodomethoxy, dichloromethoxy, trichloromethoxy, difluoromethoxy, trifluoromethoxy, chlorodifluoromethoxy, bromodifluoromethoxy, dichlorofluoromethoxy, 1-fluoroethoxy, 2-fluoroethoxy, 2-chloroethoxy, 2-bromoethoxy, 2-iodoethoxy, 2,2,2-trifluoroethoxy, 2,2,2-trichloroethoxy, pentafluoroethoxy, 1-fluoroisopropoxy, 3-fluoropropoxy, 3-chloropropoxy, 3-bromopropoxy, 4-fluorobutoxy, 4-chlorobutoxy, and like groups.

Examples of the C₁₋₄ alkylthio group include linear or branched alkylthio groups having 1 to 4 carbon atoms, such as methylthio, ethylthio, n-propylthio, isopropylthio, and tert-butylthio.

Examples of the C₂₋₇ alkylene group include ethylene, trimethylene, tetramethylene, pentamethylene, hexamethylene, heptamethylene, and the like. These alkylene groups may contain an optionally substituted nitrogen, oxygen, or sulfur atom, or a phenylene group. Examples of such alkylene groups include —CH₂NHCH₂—, —CH₂NHCH₂CH₂—, —CH₂NHNHCH₂—, —CH₂CH₂NHCH₂CH₂—, —CH₂NHNHCH₂CH₂—, —CH₂NHCH₂NHCH₂—, —CH₂CH₂CH₂NHCH₂CH₂CH₂—, —CH₂OCH₂CH₂—, —CH₂CH₂OCH₂CH₂—, —CH₂SCH₂CH₂—, —CH₂CH₂SCH₂CH₂—,

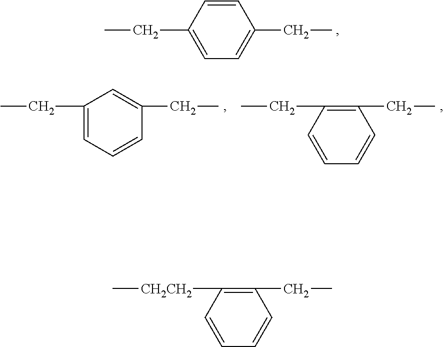

and like groups. These alkylene groups may be substituted at any position or on the nitrogen atom. Examples of such substituents include C₁₋₄ alkyl, C₁₋₆ alkoxycarbonyl, hydroxy, and like groups.

Examples of the C₁₋₄ alkylcarbonyl group include linear or branched alkylcarbonyl groups having 1 to 4 carbon atoms, such as methylcarbonyl (acetyl), ethylcarbonyl (propionyl), n-propylcarbonyl (butyryl), isopropylcarbonyl (isobutyryl), n-butylcarbonyl (valeryl), isobutylcarbonyl (isovaleryl), sec-butylcarbonyl, and tert-butylcarbonyl.

Examples of the mono- or di(C₁₋₄ alkyl)aminocarbonyl group include alkylaminocarbonyl groups in which nitrogen atoms of the aminocarbonyl groups are mono- or di-substituted with linear or branched alkyl groups having 1 to 4 carbon atoms, such as methylaminocarbonyl, dimethylaminocarbonyl, ethylaminocarbonyl, methylethylaminocarbonyl, diethylaminocarbonyl, n-propylaminocarbonyl, isopropylaminocarbonyl, n-butylaminocarbonyl, sec-butylaminocarbonyl, tert-butylaminocarbonyl, and dibutylaminocarbonyl.

Examples of the hydroxyalkyl group include linear or branched alkyl groups having 1 to 4 carbon atoms and substituted with 1 or 2 hydroxy groups, such as hydroxymethyl, 2-hydroxyethyl, 1-hydroxy-2-propyl, 3-hydroxypropyl, 4-hydroxybutyl, and 3,4-dihydroxybutyl.

The N-pyridylpiperidine compound represented by Formula (1) includes N-pyridylpiperidine compounds represented by the following Formulas (1a), (1b), and (1c):

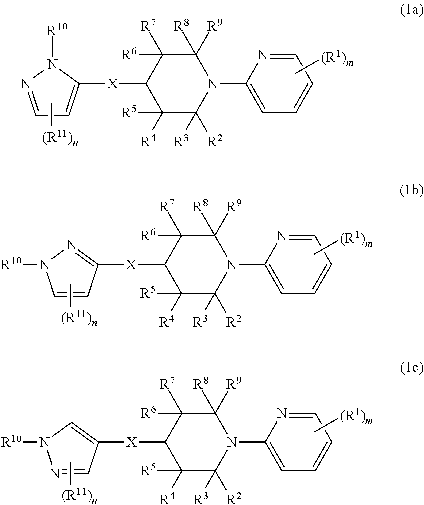

wherein R¹, R², R³, R⁴, R⁵, R⁶, R⁷, R⁸, R⁹, R¹⁰, R¹¹, X, m, and n are as defined above.

The N-pyridylpiperidine compound of Formula (1), wherein R² and R⁸ join to form a C₁₋₄ alkylene group may exist as, for example, cis-trans isomers represented by the following Formulas (1d) and (1e). The N-pyridylpiperidine compound of the invention represented by Formula (1) includes such isomers.

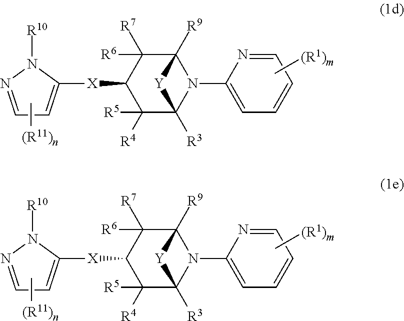

wherein R¹, R³, R⁴, R⁵, R⁶, R⁷, R⁹, R¹⁰, R¹¹, X, m, and n are as defined above, and Y is a C₁₋₄ alkylene group.

The N-pyridylpiperidine compound of Formula (1), wherein R⁴ and R⁶ join to form a C₁₋₄ alkylene group may exist as, for example, cis-trans isomers represented by the following Formulas (1f) and (1g). The N-pyridylpiperidine compound of the invention represented by Formula (1) includes such isomers.

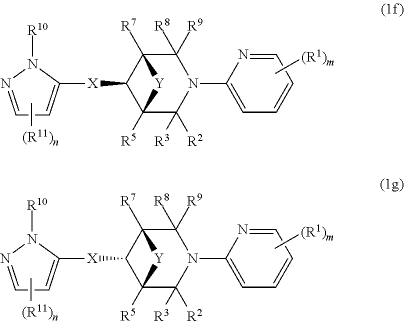

wherein R¹, R², R³, R⁵, R⁷, R⁸, R⁹, R¹⁰, R¹¹, X, m, and n are as defined above.

The N-pyridylpiperidine compound of Formula (1), wherein at least one of R², R³, R⁴, R⁵, R⁶, R⁷, R⁸, and R⁹ is a C₁₋₄ alkyl group may exist as stereoisomers in relation to the 4-position of the piperidine ring. The N-pyridylpiperidine compound of the invention represented by Formula (1) includes such isomers.

The N-pyridylpiperidine compound represented by Formula (1) may exist as N-oxides formed by oxidation of the nitrogen atom of the pyridine ring or piperidine ring of the N-pyridylpiperidine compound. The N-pyridylpiperidine compound of the invention represented by Formula (1) includes these N-oxides.

In this specification, for convenience, N-oxide formed by oxidation of the nitrogen atom on the pyridine ring is called N-pyridyl oxide, whereas N-oxide formed by oxidation of the nitrogen atom on the piperidine ring is called N-piperidyl oxide.

The N-pyridylpiperidine compound represented by Formula (1) has basic properties, and therefore can form salts with, for example, inorganic acids, such as hydrochloric acid, sulfuric acid, and phosphoric acid; organic acids, such as formic acid, acetic acid, fumaric acid, oxalic acid, and sulfonic acid; and acid salts, such as sodium hydrogen sulfate and potassium hydrogen sulfate. The N-pyridylpiperidine compound of the invention represented by Formula (1) includes these salts.

Among the N-pyridylpiperidine compounds represented by Formula (1), those wherein R¹ is a C₁₋₄ haloalkyl group, a cyano group, or a nitro group are preferable, and those wherein R¹ is a C₁₋₄ haloalkyl group are more preferable. Specifically, those wherein R¹ is a trifluoromethyl group are particularly preferable.

Preferable among the N-pyridylpiperidine compounds represented by Formula (1) are those wherein R¹⁰ is a C₁₋₂₀ alkyl group; a C₂₋₆ alkenyl group; a C₁₋₆ haloalkyl group; a C₁₋₆ alkylcarbonyl group; a phenyl group (optionally substituted on the phenyl ring with one or more, and preferably one or two substituents each independently selected from the group consisting of halogen, C₁₋₄ alkyl, and C₁₋₄ haloalkyl); a heterocyclic group (optionally substituted on the heterocyclic ring with one or more, and preferably one or two substituents each independently selected from the group consisting of C₁₋₄ alkyl and C₁₋₄ haloalkyl); or a C₁₋₄ alkyl group substituted with one or more, and preferably one or two substituents each independently selected from the group consisting of C₁₋₆ alkoxy, phenyl (optionally substituted on the phenyl ring with one or more, and preferably one or two halogen atoms), and heterocyclic groups. More preferable are those wherein R¹⁰ is a C₁₋₆ alkyl group; a C₂₋₆ alkenyl group; a phenyl group (optionally substituted on the phenyl ring with one or more, and preferably one or two halogen atoms or C₁₋₄ alkyl groups); a pyridyl group (optionally substituted on the pyridine ring with one or more, and preferably one or two C₁₋₄ alkyl groups); or a C₁₋₄ alkyl group substituted with one or two substituents each independently selected from the group consisting of C₁₋₆ alkoxy, phenyl (optionally substituted on the phenyl ring with one or more, and preferably one or two halogen atoms), and 1,3-dioxolan-2-yl. Particularly preferable are the compounds wherein R¹⁰ is a C₁₋₆ alkyl group, a pyridyl group, a 2,2-dimethoxyethyl group, or a (1,3-dioxolan-2-yl)methyl group.

Preferable among the N-pyridylpiperidine compounds of the invention represented by Formula (1) are those wherein R¹¹ is a C₁₋₆ alkyl group, a C₁₋₄ haloalkyl group, a phenyl group (optionally substituted on the phenyl ring with one or more, and preferably one to three substituents each independently selected from the group consisting of halogen, C₁₋₄ alkyl, nitro, C₁₋₄ haloalkyl, and C₁₋₄ haloalkoxy), or a heterocyclic group (optionally substituted on the heterocyclic ring with one or more, and preferably one or two halogen atoms). More preferable are those wherein R¹¹ is a trifluoromethyl group or a phenyl group (optionally substituted on the phenyl ring with one to three halogen atoms).

Preferable among the N-pyridylpiperidine compounds of the invention represented by Formula (1) are those wherein X is an oxygen atom.

More preferable are compounds of Formula (1) wherein R¹ is a C₁₋₄ haloalkyl group, a cyano group, or a nitro group; R¹⁰ is a C₁₋₂₀ alkyl group; a C₂₋₆ alkenyl group; a C₁₋₆ haloalkyl group; a C₁₋₆ alkylcarbonyl group; a phenyl group (optionally substituted on the phenyl ring with one or more, and preferably one or two substituents each independently selected from the group consisting of halogen, C₁₋₄ alkyl, and C₁₋₄ haloalkyl); a heterocyclic group (optionally substituted on the heterocyclic ring with one or more, and preferably one or two substituents each independently selected from the group consisting of C₁₋₄ alkyl and C₁₋₄ haloalkyl); or a C₁₋₄ alkyl group substituted with one or more, and preferably one or two substituents each independently selected from the group consisting of C₁₋₆ alkoxy, phenyl (optionally substituted on the phenyl ring with one or more, and preferably one or two halogen atoms), and heterocyclic groups; R¹¹ is a C₁₋₆ alkyl group, a C₁₋₄ haloalkyl group, a phenyl group (optionally substituted on the phenyl ring with one or more, and preferably one to three substituents each independently selected from the group consisting of halogen, C₁₋₄ alkyl, nitro, C₁₋₄ haloalkyl, and C₁₋₄ haloalkoxy), or a heterocyclic group (optionally substituted on the heterocyclic ring with one or more, and preferably one or two halogen atoms); and X is an oxygen atom.

Among these preferable compounds, particularly preferable are those wherein R¹ is a C₁₋₄ haloalkyl group; R¹⁰ is a C₁₋₆ alkyl group; a C₂₋₆ alkenyl group; a phenyl group (optionally substituted on the phenyl ring with one or more, and preferably one or two halogen atoms or C₁₋₄ alkyl groups); a pyridyl group (optionally substituted on the pyridine ring with one or more C₁₋₄ alkyl groups); or a C₁₋₄ alkyl group substituted with one or two substituents each independently selected from the group consisting of C₁₋₄ alkoxy, phenyl (optionally substituted on the phenyl ring with one or more, and preferably one or two halogen atoms), and 1,3-dioxolan-2-yl; R¹¹ is a trifluoromethyl group or a phenyl group (optionally substituted on the phenyl ring with one to three halogen atoms); and X is an oxygen atom.

Among the N-pyridylpiperidine compounds of the invention represented by Formula (1), those represented by Formulas (1a), (1b), and (1f) are preferable, and those represented by Formulas (1a) and (1f) are more preferable.

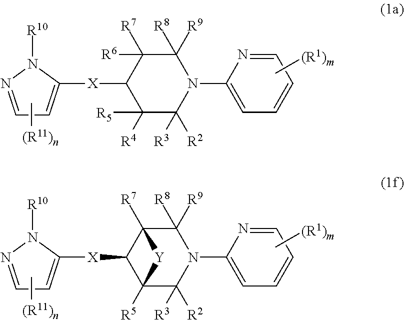

wherein R¹, R², R³, R⁵, R⁷, R⁸, R⁹, R¹⁰, R¹¹, X, m, and n are as defined above.

Among the N-pyridylpiperidine compounds of the invention represented by Formulas (1a) and (1f), those wherein R¹ is a C₁₋₄ haloalkyl group or a cyano group are preferable, and those wherein R¹ is a C₁₋₄ haloalkyl group are more preferable. Specifically, the compounds wherein R¹ is a trifluoromethyl group are particularly preferable.

Among the N-pyridylpiperidine compounds of the invention represented by Formulas (1a) and (1f), preferable are those wherein R¹⁰ is a C₁₋₂₀ alkyl group; a C₂₋₆ alkenyl group; a C₁₋₆ haloalkyl group; a C₁₋₆ alkylcarbonyl group; a phenyl group (optionally substituted on the phenyl ring with one or more, and preferably one or two substituents each independently selected from the group consisting of halogen, C₁₋₄ alkyl, and C₁₋₄ haloalkyl); a heterocyclic group (optionally substituted on the heterocyclic ring with one or more, and preferably one or two substituents each independently selected from the group consisting of C₁₋₄ alkyl and C₁₋₄ haloalkyl); or a C₁₋₄ alkyl group substituted with one or more, and preferably one or two substituents each independently selected from the group consisting of C₁₋₆ alkoxy, phenyl (optionally substituted on the phenyl ring with one or more, and preferably one or two halogen atoms), and heterocyclic groups. More preferable are those wherein R¹⁰ is a C₁₋₆ alkyl group; a C₂₋₆ alkenyl group; a phenyl group (optionally substituted on the phenyl ring with one or more, and preferably one or two halogen atoms or C₁₋₄ alkyl groups); a pyridyl group (optionally substituted on the pyridine ring with one or more, and preferably one or two C₁₋₄ alkyl groups); or a C₁₋₄ alkyl group substituted with one or two substituents each independently selected from the group consisting of C₁₋₆ alkoxy, phenyl (optionally substituted on the phenyl ring with one or more, and preferably one or two halogen atoms), and 1,3-dioxolan-2-yl. Particularly preferable are compounds wherein R¹⁰ is a C₁₋₆ alkyl group, a pyridyl group, a 2,2-dimethoxyethyl group, or a (1,3-dioxolan-2-yl)methyl.

Among the N-pyridylpiperidine compounds of the invention represented by Formulas (1a) and (1f), preferable are those wherein R¹¹ is a C₁₋₆ alkyl group, a C₁₋₄ haloalkyl group, a phenyl group (optionally substituted on the phenyl ring with one or more, and preferably one to three substituents each independently selected from the group consisting of halogen, C₁₋₄ alkyl, nitro, C₁₋₄ haloalkyl, and C₁₋₄ haloalkoxy), or a heterocyclic group (optionally substituted on the heterocyclic ring with one or more, and preferably one or two halogen atoms). More preferable are compounds wherein R¹¹ is a trifluoromethyl group or a phenyl group (optionally substituted on the phenyl ring with one to three halogen atoms).

Among the N-pyridylpiperidine compounds of the invention represented by Formulas (1a) and (1f), those wherein X is an oxygen atom are preferable.

More preferable are compounds of Formulas (1a) and (1f) wherein R¹ is a C₁₋₄ haloalkyl group or a cyano group; R¹⁰ is a C₁₋₂₀ alkyl-group; a C₂₋₆ alkenyl group; a C₁₋₆ haloalkyl group; a C₁₋₆ alkylcarbonyl group; a phenyl group (optionally substituted on the phenyl ring with one or more, and preferably one or two substituents each independently selected from the group consisting of halogen, C₁₋₄ alkyl, and C₁₋₄ haloalkyl); a heterocyclic group (optionally substituted on the heterocyclic ring with one or more, and preferably one or two substituents each independently selected from the group consisting of C₁₋₄ alkyl and C₁₋₄ haloalkyl); or a C₁₋₄ alkyl group substituted with one or more, and preferably one or two substituents each independently selected from the group consisting of C₁₋₆ alkoxy, phenyl (optionally substituted on the phenyl ring with one or more, and preferably one or two halogen atoms), and heterocyclic groups; R¹¹ is a C₁₋₆ alkyl group, a C₁₋₄ haloalkyl group, a phenyl group (optionally substituted on the phenyl ring with one or more, and preferably 1 to 3 substituents each independently selected from the group consisting of halogen, C₁₋₄ alkyl, nitro, C₁₋₄ haloalkyl, and C₁₋₄ haloalkoxy), or a heterocyclic group (optionally substituted on the heterocyclic ring with one or more, and preferably one or two halogen atoms); and X is an oxygen atom.

Among these preferable compounds, particularly preferable are those wherein R¹ is a C₁₋₄ haloalkyl group, R¹⁰ is a C₁₋₆ alkyl group; a C₂₋₆ alkenyl group; a phenyl group (optionally substituted on the phenyl ring with one or more, and preferably one or two halogen atoms or C₁₋₄ alkyl groups); a pyridyl group (optionally substituted on the pyridine ring with one or more, and preferably one or two C₁₋₄ alkyl groups); or a C₁₋₄ alkyl group substituted with one or two substituents each independently selected from the group consisting of C₁₋₆ alkoxy, phenyl (optionally substituted on the phenyl ring with one or more, and preferably one or two halogen atoms), and 1,3-dioxolan-2-yl; R¹¹ is a trifluoromethyl group or a phenyl group (optionally substituted on the phenyl ring with one to three halogen atoms); and X is an oxygen atom.

Among the N-pyridylpiperidine compounds of the invention represented by Formula (1a), preferable are those wherein any one of R⁴, R⁵, R⁶, and R⁷ is a C₁₋₄ alkyl group that is positioned trans to the X on the 4-position of the piperidine ring. Particularly preferable are compounds wherein the C₁₋₄ alkyl group is a methyl group.

### Method of Producing N-pyridylpiperidine Compound

The N-pyridylpiperidine compound represented by Formula (1) can be produced, for example, by the method described in WO 2008/026658.

### Ectoparasite-Controlling Agent

The animal ectoparasite-controlling agent of the present invention characteristically comprises the N-pyridylpiperidine compound represented by Formula (1) as an active ingredient.

The controlling agent of the present invention is effective against fleas, mites, lice (cattle lice, horse lice, sheep lice, linognathus vituli, head lice, etc.), biting lice (Trichodectes canis, etc.), and the like that live in the body surface of host animals. In particular, the controlling agent of the present invention has the beneficial effect of preventing mites. In addition, the controlling agent of the present invention is effective against blood-sucking dipteran insects, such as flies, biting midges, black flies, and stable flies.

Fleas refer to ectoparasitic wingless insects belonging to Siphonaptera, specifically fleas belonging to Pulicidae, Ceratophyllus, or the like. Examples of fleas belonging to Pulicidae include Ctenocephalides canis, Ctenocephalides felis, Pulex irritans, Echidnophaga gallinacea, Xenopsylla cheopis, Monopsyllus anisus, Nosopsyllus fasciatus, etc.

Mites are, for example, ticks. Examples thereof include Haemaphysalis longicornis, Haemaphysalis japonica, Dermacentor reticulatus, Dermacentor taiwanesis, Haemaphysalis flava, Ixodes ovatus, Ixodes persulcatus, Boophilus microplus, etc.

Examples of host animals for which the controlling agent of the present invention is effective include pets, such as dogs, cats, mice, rats, hamsters, guinea pigs, squirrels, rabbits, ferrets, and birds (e.g., pigeons, parrots, myna birds, paddy birds, parakeets, lovebirds, and canaries); livestock, such as cattle, horses, pigs, and sheep; poultry, such as ducks and chicken; and the like. Ectoparasites are parasitic and live on the back, infra-axillary region, lower abdominal region, inner thigh region, etc., of these host animals.

The controlling agent of the present invention may be used as it is, without the addition of any other components. Alternatively, the controlling agent can be mixed with various suitable carriers in the form of liquids, solids, or gases, optionally followed by addition of surfactants and other auxiliary materials for preparation of formulations, and then formulated into granules, fine granules, tablets, powders, capsules, premix formulations, solutions, emulsions, and other dosage forms.

The amount of the compound of the present invention as an active ingredient in such formulations can be suitably selected from a wide range, depending on various conditions including the type of formulation, place of application, etc. Such formulations usually contain the compound in an amount of about 0.01 to 95 wt. %, and preferably about 0.1 to 50 wt. %.

The aforementioned suitable carriers may be those generally used in animal feed drugs. Examples thereof are lactose, sucrose, glucose, starch, wheat flour, corn flour, soybean oil cake, defatted rice bran, calcium carbonate, and other commercially available feed raw materials.

Examples of the surfactant include anionic surfactants (e.g., alkali stearate, sodium abietate, alkyl sulfate, sodium dodecylbenzenesulfonate, sodium dioctylsulfosuccinate, and fatty acids), cationic surfactants (e.g., water-soluble quaternary ammonium), nonionic surfactants (optionally selected from polyoxyethylenated sorbitan esters, polyoxyethylenated alkyl ethers, polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide, etc.), amphoteric surfactants (e.g., lauryl-substituted betaine compounds), and the like.

Examples of auxiliary materials for preparation of formulations include fixing agents, dispersing agents, thickeners, preservatives, anti-freezing agents, stabilizers, adjuvants, and the like.

Examples of fixing agents and dispersing agents include casein, gelatin, polysaccharides (e.g., starch, gum arabic, cellulose derivatives, and alginic acid), lignin derivatives, bentonite, sugars, water-soluble synthetic polymers (e.g., polyvinyl alcohol, polyvinylpyrrolidone, and polyacrylic acids), and the like.

Examples of thickeners include water-soluble polymer compounds, such as xanthan gum and carboxymethyl cellulose, high-purity bentonite, white carbon, and the like.

Examples of preservatives include sodium benzoate, p-hydroxybenzoic acid ester, and the like.

Examples of anti-freezing agents include ethylene glycol, diethylene glycol, and the like.

Examples of stabilizers include PAP (acidic isopropyl phosphate), BHT (2,6-di-tert-butyl-4-methylphenol), BHA (a mixture of 2-tert-butyl-4-methoxyphenol and 3-tert-butyl-4-methoxyphenol), vegetable oils, mineral oils, surfactants, fatty acids and esters thereof, and the like.

Examples of adjuvants include soybean oil, corn oil, and like vegetable oils, machine oil, glycerin, polyethylene glycol, and the like.

Such formulations may be colored with an organic or inorganic dye.

The thus-obtained formulations can be used as they are or after being diluted with water or the like. However, fine granules, granules, etc., are generally used as they are, without being diluted. When emulsions, wettable powders, flowable formulations, etc., are used after being diluted with water or the like, the active ingredient concentration is generally 0.0001 to 50 wt. %, and preferably about 0.001 to 10 wt. %.

In addition, the controlling agent of the present invention may be previously mixed with other agents, such as insecticides, nematocides, acaricides, fungicides, antifungals, antibacterial agents, anti-inflammatory agents, antiprotozoan drugs, synergists (e.g., piperonyl butoxide), or the like, and then formulated. Alternatively, the formulations of the present invention and other such agents may be used in combination when used.

When the controlling agent of the present invention is mixed with other animal drugs, the proportion of N-pyridylpiperidine compound and other animal drugs is not particularly limited, but is generally 100:0 to 1:99 (weight ratio).

Although the dose of the controlling agent of the present invention varies depending on the administration method, the purpose of administration, disease symptoms, etc., the controlling agent of the present invention may generally be administered to a host animal in a dose of 0.01 mg or more and 100 g or less, and preferably 0.1 mg or more and 10 g or less, per kg of body weight of the host animal.

The controlling agent of the present invention is orally or parenterally administered to a host.

When orally administered, for example, the controlling agent of the present invention is mixed with feed of a host animal, and then administered together with the feed; or tablets, solutions, capsules, wafers, biscuits, minced meat, etc., containing the controlling agent of the present invention are administered.

When parenterally administered, for example, the controlling agent of the present invention is formed into suitable formulations, and then incorporated into the body by intravenous infusion administration, intramuscular administration, intracutaneous administration, subcutaneous administration, spot-on treatment, pore-on treatment, or the like; or resin pieces, etc., containing the controlling agent of the present invention are implanted under the skin of a host animal.

### EXAMPLES

The present invention is described in more detail below with reference to test examples of the controlling agent of the present invention; however, the present invention is not limited thereto.

### Test Example: Mortality of Ixodid Ticks by Filter Paper Clipping Method

### (1) Preparation of Drug

Among the compounds disclosed in WO 2008/026658, Compound Nos. 1a-16, 1a-17, 1a-62, 1a-75, 1a-76, 1a-174, 1a-201, 1a-208, 1a-234, 1a-251, 1a-262, 1a-267, 1a-268, 1a-274, 1a-302, 1f-38, and 1f-39 were used as Test Compounds 1 to 17.

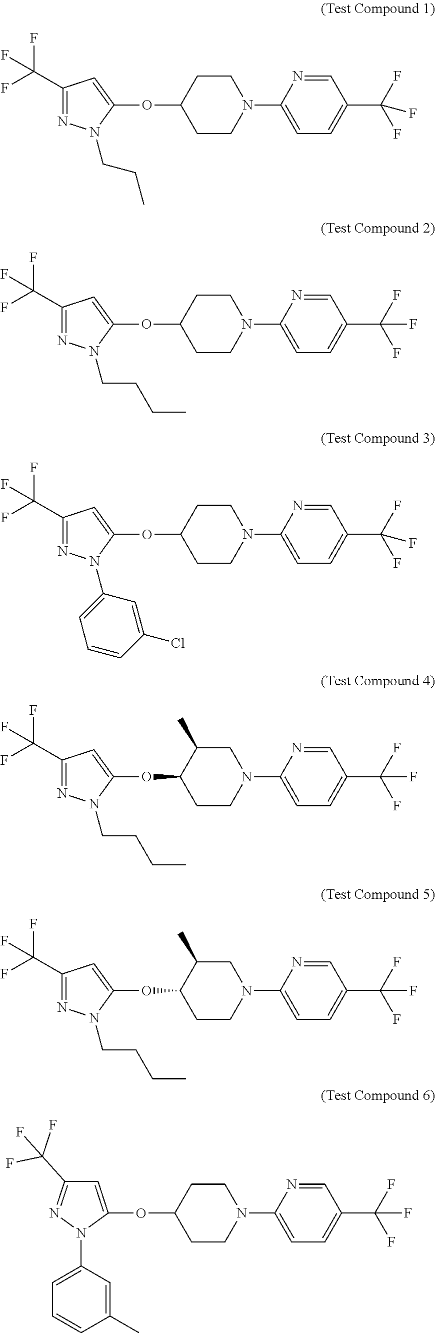 [SMILES: CCCn1nc(C(F)(F)F)cc1OC1CCN(c2ccc(C(F)(F)F)cn2)CC1 ; CCCCn1nc(C(F)(F)F)cc1OC1CCN(c2ccc(C(F)(F)F)cn2)CC1 ; FC(F)(F)c1ccc(N2CCC(Oc3cc(C(F)(F)F)nn3-c3cccc(Cl)c3)CC2)nc1 ; CCCCn1nc(C(F)(F)F)cc1O[C@@H]1CCN(c2ccc(C(F)(F)F)cn2)C[C@@H]1C ; CCCCn1nc(C(F)(F)F)cc1O[C@H]1CCN(c2ccc(C(F)(F)F)cn2)C[C@@H]1C ; Cc1cccc(-n2nc(C(F)(F)F)cc2OC2CCN(c3ccc(C(F)(F)F)cn3)CC2)c1]

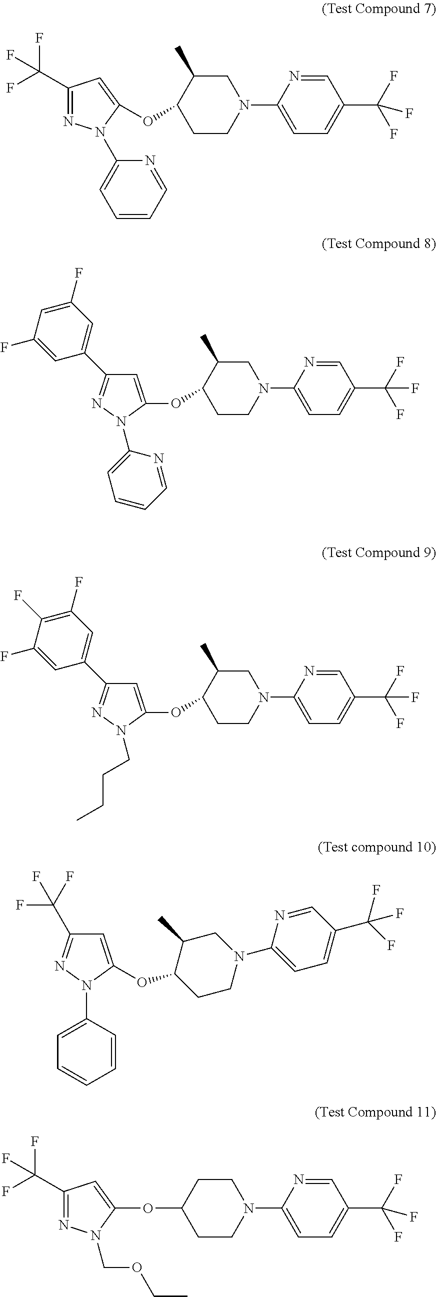 [SMILES: C[C@H]1CN(c2ccc(C(F)(F)F)cn2)CC[C@@H]1Oc1cc(C(F)(F)F)nn1-c1ccccn1 ; C[C@H]1CN(c2ccc(C(F)(F)F)cn2)CC[C@@H]1Oc1cc(-c2cc(F)cc(F)c2)nn1-c1ccccn1 ; CCCCn1nc(-c2cc(F)c(F)c(F)c2)cc1O[C@H]1CCN(c2ccc(C(F)(F)F)cn2)C[C@@H]1C ; C[C@H]1CN(c2ccc(C(F)(F)F)cn2)CC[C@@H]1Oc1cc(C(F)(F)F)nn1-c1ccccc1 ; CCOCn1nc(C(F)(F)F)cc1OC1CCN(c2ccc(C(F)(F)F)cn2)CC1]

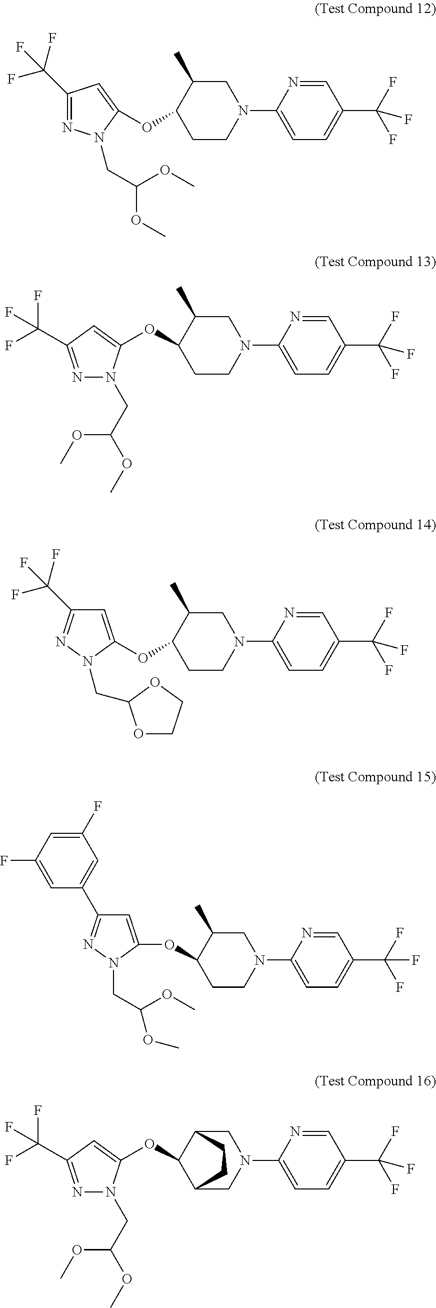

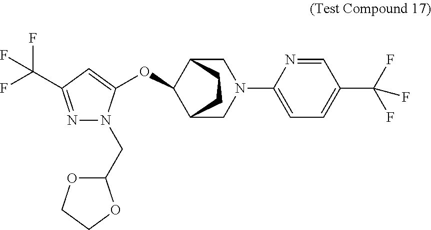

Acetone was added to each of Test Compounds 1 to 17 so that the concentration was 0.5 mg/ml, thereby preparing solutions. Although Test Compound 15, which was not dissolved in acetone, formed a heterogeneous suspension, the suspension was used as it was.

### (2) Filter Paper Clipping Method

Each of the above prepared solutions was added dropwise in an amount of 1 ml to a square filter paper (5×10 cm; area: 50 cm²), and dried on aluminum foil at room temperature for 24 hours. Then, each filter paper was folded double on the long side, and both sides were secured with bulldog clips into a bag shape. About 20 ixodid ticks were placed in the bag-like filter paper, and the opening was sealed with a bulldog clip. After 72 hours, the number of dead ticks was calculated. Thereafter, the surviving ticks were killed in a freezer, and the total number of ticks was calculated.

As a result, a mortality of 70% or more was achieved by all of Test Compounds 1 to 17.

1. An animal ectoparasite-controlling agent comprising an N-pyridylpiperidine compound, an N-oxide thereof, or salts of these compounds, the N-pyridylpiperidine compound being represented by Formula (1): 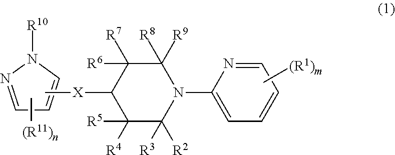
wherein R¹ is a halogen atom, a C₁₋₄ haloalkyl group, a cyano group, a nitro group, or a C₁₋₄ alkoxycarbonyl group;
R², R³, R⁴, R⁵, R⁶, R⁷, R⁸, and R⁹ are each a hydrogen atom or a C₁₋₄ alkyl group;
each pair of R² and R⁸, and R⁴ and R⁶, may join to form a C₁₋₄ alkylene group;
R¹⁰ is a hydrogen atom; a C₁₋₂₀ alkyl group; a C₃₋₈ cycloalkyl group; a C₂₋₆ alkenyl group; a C₂₋₆ alkynyl group; a C₁₋₆ haloalkyl group; a C₂₋₆ haloalkenyl group; a C₁₋₆ alkylcarbonyl group; a C₁₋₆ alkoxycarbonyl group; a benzoyl group optionally substituted on the phenyl ring with one to five halogen atoms; a phenyl group optionally substituted on the phenyl ring with one or more substituents each independently selected from the group consisting of halogen, C₁₋₄ alkyl, and C₁₋₄ haloalkyl; a heterocyclic group optionally substituted on the heterocyclic ring with one or more substituents each independently selected from the group consisting of halogen, C₁₋₄ alkyl, C₁₋₄ haloalkyl, and optionally substituted heterocyclic groups; or a C₁₋₄ alkyl group optionally substituted with one or more substituents each independently selected from the group consisting of optionally halogen-substituted C₃₋₈ cycloalkyl, cyano, nitro, formyl, C₁₋₆ alkoxy, C₁₋₄ haloalkoxy, benzyloxy, phenoxy, —CON(R¹²)(R¹³), phenyl optionally substituted on the phenyl ring with one or more halogen atoms, and heterocyclic groups optionally substituted on the heterocyclic ring with one or more C₁₋₄ alkyl groups; wherein R¹² and R¹³ are each a C₁₋₄ alkyl group, or R¹² and R¹³ may join to form a C₂₋₇ alkylene group;
R¹¹ is a halogen atom; a C₁₋₆ alkyl group; a C₁₋₄ haloalkyl group; a C₁₋₄ hydroxyalkyl group; a C₁₋₄ alkoxycarbonyl group; a C₁₋₄ alkylcarbonyl group; a mono or di(C₁₋₄ alkyl)aminocarbonyl group; a nitro group; a cyano group; a formyl group; —C(R¹⁴)═NO(R¹⁵); a phenyl group optionally substituted on the phenyl ring with one or more substituents each independently selected from the group consisting of halogen, C₁₋₆ alkyl, C₁₋₄ haloalkyl, C₁₋₆ alkoxy, C₁₋₄ haloalkoxy, C₁₋₄ alkylthio, cyano, and nitro; or a heterocyclic group optionally substituted on the heterocyclic ring with one or more substituents each independently selected from the group consisting of halogen, C₁₋₄ alkyl, and C₁₋₄ haloalkyl; wherein R¹⁴ is a hydrogen atom or a C₁₋₄ alkyl group, and R¹⁵ is a hydrogen atom, a C₁₋₄ alkyl group, or a benzyl group;
X is an oxygen atom, a sulfur atom, or —SO₂—;
m is an integer of 1 to 4, and when m is an integer of 2 or more, the R¹'s, the number of which is represented by m, may be the same or different; and
n is an integer of 1 or 2, and when n is 2, the two R¹¹'s may be the same or different.

2. The animal ectoparasite-controlling agent according to claim 1, wherein the N-pyridylpiperidine compound is represented by Formula (1) in which R¹ is a halogen atom, a C₁₋₄ haloalkyl group, a cyano group, or a nitro group.

3. The animal ectoparasite-controlling agent according to claim 1, wherein the N-pyridylpiperidine compound is represented by Formula (1) in which R¹⁰ is a hydrogen atom; a C₁₋₂₀ alkyl group; a C₂₋₆ alkenyl group; a C₁₋₆ haloalkyl group; a C₁₋₆ alkylcarbonyl group; a C₁₋₆ alkoxycarbonyl group; a benzoyl group optionally substituted on the phenyl ring with one to five halogen atoms; a phenyl group optionally substituted on the phenyl ring with one or more substituents each independently selected from the group consisting of halogen, C₁₋₄ alkyl, and C₁₋₄ haloalkyl; a heterocyclic group optionally substituted on the heterocyclic ring with one or more substituents each independently selected from the group consisting of halogen, C₁₋₄ alkyl, C₁₋₄ haloalkyl, and optionally substituted heterocyclic groups; or a C₁₋₄ alkyl group substituted with one or more substituents each independently selected from the group consisting of formyl, C₁₋₆ alkoxy, phenyl optionally substituted on the phenyl ring with one or more halogen atoms, and heterocyclic groups optionally substituted on the heterocyclic ring with one or more C₁₋₄ alkyl groups.

4. The animal ectoparasite-controlling agent according to claim 1, wherein the N-pyridylpiperidine compound is represented by Formula (1) in which R¹¹ is a halogen atom; a C₁₋₆ alkyl group; a C₁₋₄ haloalkyl group; a C₁₋₄ hydroxyalkyl group; a C₁₋₄ alkoxycarbonyl group; a formyl group; —C(R¹⁴)═NO(R¹⁵) wherein R¹⁴ is a hydrogen atom, and R¹⁵ is a hydrogen atom or a C₁₋₄ alkyl group; a phenyl group optionally substituted on the phenyl ring with one or more substituents each independently selected from the group consisting of halogen, C₁₋₆ alkyl, C₁₋₄ haloalkyl, C₁₋₆ alkoxy, C₁₋₄ haloalkoxy, C₁₋₄ alkylthio, cyano, and nitro; or a heterocyclic group optionally substituted on the heterocyclic ring with one or more halogen atoms.

5. The animal ectoparasite-controlling agent according to claim 1, wherein the N-pyridylpiperidine compound is represented by Formula (1) in which X is an oxygen atom.

6. A method for preventing or treating infection in an animal caused by parasites, the method comprising administrating the animal ectoparasite-controlling agent according to claim 1 to the animal.
